(19) United States
(12) Patent Application Publication
(10) Pub. No.: US 2003/0195543 A1
(43) Pub. Date: Oct. 16, 2003

(54) Ear piercing healing grommet system

(76) Inventor: Steven Danno, Laguna Woods, CA (US)

Correspondence Address: MATTHEW A. NEWBOLES, STETINA BRUNDA GARRED & BRUCKER, 75 Enterprise, Suite 250, Aliso Viejo, CA, 92656, US

(21) Appl. No.: 10/441,769

(22) Filed: May 20, 2003

Related U.S. Application Data
(63) Continuation-in-part of application No. 09/747,128, filed on Dec. 22, 2000, now abandoned.
Application No. 09/747,128 is a continuation-in-part of application No. 09/264,498, filed on Mar. 8, 1999, now Pat. No. 6,183,490.

(51) Int. Cl. A61B 17/34

(52) U.S. Cl. 606/188

(57) ABSTRACT

A healing grommet and system for facilitating the healing of a piercing via use of a healing grommet. According to a preferred embodiment, the grommet is comprised of an elongate sleeve positionable within a piercing formed through tissue. The lumen is operative to releasably engage an item of jewelry, and in particular an earring, therein to thus enable jewelry to be replaced while the healing grommet facilitates healing for the piercing.

### CROSS-REFERENCE TO RELATED APPLICATIONS

This Application is a continuation-in-part of U.S. patent application Ser. No. 09/747,128 filed Dec. 22, 2000 which is a continuation-in-part of U.S. patent application Ser. No. 09/264,498 filed Mar. 8, 1999.

### STATEMENT RE: FEDERALLY SPONSORED RESEARCH/DEVELOPMENT

Not Applicable

### BACKGROUND OF THE INVENTION

It is well known and well publicized that infections and problems associated with ear piercing continue to be widespread as they have remained so for decades. It is additionally well known in the industry that the sale of jewelry is a primary object of the dominant vendors engaged in such business, and that the ear piercing service is a means to the primary end insofar as the piercing service is frequently offered “free” with the purchase of jewelry.

While the pierced openings of most end user consumers eventually heal, it is well known that a significant number of piercings do not heal optimally, and that a significant number sustain unnecessarily long term recurring discomfort traceable to the original piercing. It is appreciated that a piercing, and ear piercing in particular, involves the formation of a new epidermal passage through flesh and requires extraordinary care and maintenance throughout an advisable eight-week healing phase. Such care presents an extraordinary challenge to young consumers. In fact it is well known that the frequent reluctance of young consumers to follow proper care procedures which contributes mainly to the cause of such piercing infections and problems. Given the absence of viable choice in the marketplace, consumers, many of which are young children, often must accept the only standard of service available. Such reluctant participation even occurs despite the fact that parents remain extremely suspicious and concerned about the quality of piercing being practiced at most retail sites. Parents of increasingly younger end user consumers would greatly appreciate more choices that address their serious consumer issues and importantly more choices that affordably ensure a proper healing independent of young consumers' frequent reluctance to adhere to instructions and advice for proper care and maintenance during the extended healing phase.

It is well to understand well known industry dynamics affecting consumer behavior. One dynamic includes the marketing practices and incentives of the current piercing vendors which are frequently at cross purposes with optimum piercing and healing. It is well known that the dominant vendors have strong incentives to sell earrings, especially gold earrings with standard diameter (small diameter) posts at the point of sale and the time of piercing.

Nevertheless, The dominant industry vendors are also known to offer the above options of large diameter stainless steel piercing earrings in their product lines. Forming a larger (i.e., 0.050″ diameter) initial passageway is advantageous because the interchanging and insertion of standard (smaller diameter) earring posts (i.e., 0.028″) are then less likely to scratch or damage the still-tender walls of the pierced pathway. It is well known that young consumers are impatient to change out earrings prior to sufficient healing. It is also well known that the newly pierced pathway will readily shrink to the smaller diameter of the standard earrings in common use by consumers. However, in practice the well known market trend has been to promote the sale of the more profitable small diameter ornamental gold piercing earrings throughout the sales and distribution channels. For example, sales at the point of purchase may stall when consumers learn or are reminded that the piercing earring must remain in place for about six continual weeks, day and night. To overcome this and other consumer objections, it is well known in the industry that in order to promote the retail sale of gold earrings at the points of sale, consumers are commonly informed by retail clerks: (1) that the small diameter shaft is adequate; (2) that gold is hypoallergenic and non reactive and adequate for the healing phase; (3) that by piercing with (standard diameter) ornamental gold earring the customer can conserve their investment by having an earring they can use after piercing versus having to throw away the large diameter piercing earring; and (4) that consumers can likely change out their earrings to standard earrings earlier than is advisable (i.e., in four weeks versus six or eight weeks).

Another dynamic affecting optimal healing relates to consumer behavior, and more particularly young consumers' frequent reluctance to follow proper-care instructions. Consumers, especially younger consumers, have a strong inclination or impatience to change out earrings before healing is complete. Such impatience, combined with the above-noted tacit “permission” at the point of sale or piercing, leads too commonly to changing earrings prematurely. Invariably, while the walls of the pierced ear are still tender, consumers insert standard earrings of unknown metallic content into the small diameter opening. Frequently, the walls of the small opening are scratched, and/or the exchanged earring may not have been properly cleaned. Commonly infections and extensive delays to healing occur, not to mention other problems, often long term. A critical time period for the consumer is the last two weeks of the healing period. Hence, the strong impatience of the consumer to change out their piercing earring prior to sufficient healing is a major factor relating to infections during this period. The products and services of the dominant vendors do not adequately address the above consumer behavioral factor. As mentioned, to do so would be deemed to be at cross purposes with sale of more profitable earrings.

To maintain a relatively germ-free environment on and about the pierced incision for the recommended approximate eight week period requires extraordinary diligence frequently lacking in young consumers. For example it is well known that it is difficult for parents and guardians to persuade young people to keep their hands off any cut or wound they may have. And it is well known that young people are not in the habit of maintaining washed hands. The frequent reluctance of young consumers to adhere to proper-care advice, and the long healing period are further predictors of problem healings. A piercing system designed to comprehensively address consumer health issues cannot avoid addressing the above consumer behavioral factors. Hence, what is greatly needed in the industry is a means of completely designing around the consumers' behavioral impatience to change out earrings, and young consumers' frequent reluctance to adhere to proper care advice such as maintaining a germ-free piercing site. To be effective such a system would need to ensure optimum healing independent of and regardless of the behavior of young consumers.

As such, generally speaking a piercing/healing grommet implanted at the time of piercing would provide the means for the consumer to interchangeably insert ornamental earrings throughout the healing period. The consumer would gain every reason to be patient during the last critical weeks of healing. The tender newly pierced opening would remain insulated from any earring post introduced inside the grommet. The grommet diameter would be optimum as well, as described above. While other devices have been developed that are directed to sleeves and other like devices for facilitating the wearing of jewelry, the same are directed to use following a completely healed piercing. Exemplary of such devices include those disclosed in U.S. Pat. Nos. 4,067,341 to Ivey; 4,593,540 to Cuvar; 4,829,788 to DiDomenico; 5,018,365 to Luceno; 5,154,068 to DiDomenico; and 5,743,113 to Kogen.

For the following reasons all the referenced patents share the similar distinction of being restricted by design and intent and by word to application after the pierced channel in the ear has healed. Such insulating sleeves are either portable (Ivey) and intended to remain resident with the jewelry or they are permanently resident in a device permanently affixed to the ear (DiDomenico). On close scrutiny, and as discussed in detail below, the features desirable in such after-healed applications are mutually exclusive of the features desirable in an optimum piercing and healing devises. For example, to accommodate a permanent device for holding a sleeve DiDomenico teaches that the device should be “snugly” affixed to the ear lobe. He further emphasizes the snug fit by requiring the earring to be retained by a clasp at the back of the device. Such snug fit would be counter-productive to a device intended for optimum healing, wherein it is well known to be critical to freely rotate the shaft to prevent the attachment of serous discharge (from the incision wound) onto the foreign-body shaft. Such attachment or “bridging” is detrimental to optimal healing as it inhibits the access of new epidermal cells attempting to grow upon the incision. Likewise, the necessary snug and permanent fit of DiDemonico's and similar devices preclude the modest space and gaps needed next to the front and back of the ear to accommodate the inflow and outflow of air and liquid cleansers and ointments which are advantageous to healing.

Healing shafts need to be smooth: During healing it is critical to avoid pockets or cavities along the shaft due to the potential accumulation of bacteria. (Ideally, the material selected for the healing shaft or sleeve will be a hypoallergenic material the body will not “grow to” such as a plastic material with added elements to promote lubricity.). Yet, at least two of the patents in the above list offer shafts with such cavities coming in contact with the earlobe opening. Kogen's. And, Luceno provides for threads for threading an end cap. The threads design requires a longitudinal slot extending midway along the shaft to provide for a necessary spring tension necessarily encroach into the ear opening because Luceno recommends “abutting the earlobe front and rear”.

Healing grommets are primarily intended for use during the healing period; however, the above listed patents are admittedly designed for after healing and with permanence or semi-permanence in mind. The above products are designed to be “cleanable and re-usable” (Kogen). Healing grommets further need to be of simple design and disposable, however, most of the referenced designs are of complex and expensive construction.

The one relevant prior art known to Applicant comprises a piercing grommet that was introduced and patented by S. Thomas U.S. Pat. No. 5,411,516 issued May 2, 1995. While it may be shown to have certain drawbacks, the Thomas design serves as a revolutionary early step taken to comprehensively address and solve consumer health issues in ear piercing.

Thomas provides for a piercing and healing earring (grommet) he refers to as a hollow post which allows for the interchange of earrings throughout healing. During piercing a solid sharpened post having a sharp point is inserted into the hollow post to form one piercing assembly. Using conventional piercing guns provided by the dominant vendors, the piercing assembly is forced through the earlobe tissue, forming the desired opening. The solid post is removed and the hollow post is retained at the rear by a standard “butterfly” clutch. The hollow post (grommet) then remains implanted throughout the approximate advisable six to eight week healing phase. In the Thomas design the front of the hollow tube post is specialized to form a cup. A rubber plug with a bored opening is lodged into the cup for the purpose of receiving and frictionally holding the posts of standard earrings. Certain drawbacks of the Thomas design are noted. From a cosmetic perspective, consumers are conventionally accustomed to earrings residing adjacent to the earlobe or at least appearing to butt against the earlobe. The effect of Thomas' substantial cup/plug housing would cause the earring to stand off conspicuously from the earlobe and would be strongly unconventional and predictably undesirable to consumers. In addition Thomas teaches to frictionally retain the earring post by a “rubber plug” within a substantial “housing” formed outside the pierced opening and at the forward viewable side. Thomas teaches that his housing is multi-purpose and as such he excludes consideration of retaining the earring elsewhere. In the present invention the posts of ornamental earrings would be frictionally retained at any point or means along the bore of the grommet implant. By retaining the earring post within the grommet bore an economy of space is obtained.

Additionally, the hollow post grommet of Thomas' design is understood to be made of a very rigid material such as metal in all contemplated embodiments. A curved shaped hollow tube is not conducive to the solid carry-through post and piercing gun method described. As such, the grommet in the Thomas patent is intended to accommodate straight earrings, and not serve a dual purpose of being curved to receive and retain hoop shaped earrings. Further Thomas fails to disclose the use of antimicrobial agents with his device. And, his contemplation of a metallic piercing post and his lack of anticipating a plastic piercing post further separates Thomas' patent from anticipating the use of antimicrobial agents that may be incorporated into the device and released automatically therefrom.

Additionally Thomas makes no mention of a rear clasp that actively facilitates or optimizes healing. Thomas relies upon conventional piercing methods employed by the current dominant vendors. As such Thomas describes the common butterfly clasp as the means for retaining his “piercing earring” (implant) device. It is well known that upon applying the butterfly clasp (or any commonly used earring clasp) consumers frequently (intentionally as well as unintentionally) push the clasp past the standard detent lock channel common to most earring posts. The effect is to obtain a tighter and more secure fit of the clasp against the back of the ear. The result is that the pointed end of the earring post will protrude through the back of the butterfly clasp. Such common configuration of standard the post and clasp is counter-productive and adverse to the several optimal design features desired for an optimal clasp for piercing and healing. To name one problem, such a rear clasp configuration leaves the back of the neck behind the ear exposed to being scratched when the wearer reclines to rest or sleep.

Further Thomas incorporates the rear clasp common to conventional piercing. Such clasp is conventionally known as a “butterfly” clasp. Such clasp is invariably minimal and unspecialized. Such clasps are not designed to be cooperatively engaged with an implant shaft so they may be rotated as a unit. And, as mentioned, Thomas' multi-purpose housing limits rotation to being accomplished only by gripping and turning the forward housing. Yet rotation would also advantageously incorporate the rear clasp and be achieved by the naturally opposing forces of the thumb and index finger. Thomas' device does not intend for the rear clasp to be usefully implemented in rotation of the shaft.

It is well known in medical arts that healing of a body piercing and ear piercing in particular involves formation of new epidermal pathway along the incision track. With optimal healing new epidermal cells want to grow across the incision. The optimum conditions that promote such efficient growth is to provide a clean lubricious moist environment about the incision while occluding the wound with antibacterial ointment. Rotation of the foreign-body shaft, intended to form the new pathway, is critical to prevent the serous discharge from the wound from attaching or “bridging”. Such attachment would inhibit the access for new epidermal cells to grow over the wound. As such, any improved means of rotating the shaft would be advantageous to healing, especially given the noted frequent reluctance of young consumers to follow proper-care instruction. In addition to enhancing the lubricity of the plastic shaft, rotation may be facilitated by incorporating a specialized rear clasp (which facilitates rotation) in combination with a small forward grommet flange formed with a low silhouette.

### BRIEF SUMMARY OF THE INVENTION

The present invention specifically addresses and alleviates the above identified deficiencies in the art. In this regard, it is an object of this invention to provide an improved tubular piercing grommet implant, that remains in the ear or other body part (i.e., belly button) throughout the extended healing process, having the purpose of allowing the shafts of conventional ornamental earrings, jewelry and hoops to be interchangeably inserted immediately after piercing being frictionally retained at any point or means along the bore of the grommet.

Another object of this invention is to substantially eliminate most problems of conventional practice resulting from changing earrings or hoops before the pierced opening is fully healed. The invented piercing implant grommet will be formed of a bio-compatible material. Unlike Thomas' invention the present invention provides for an implant grommet version that may conform to the shape of the inserted ornamental earring shafts or now popular hoops. Such plastic material is further desirable during healing in that it is well known in medical arts that serous discharge from the pierced incision is much less inclined to want to attach to or “grow” to foreign objects formed of plastic versus metal. Importantly such plastic material may be saturated or coated or otherwise incorporated with active anti-microbial agents and germicides that release automatically therefrom, such release of certain types of antimicrobial agents being intentionally triggered by wetting of any kind including the ambient humidity from the epidermal incision, or from any washing of the pierced area.

Another object of the invention is to implant the grommet while piercing the ear and to provide for an optimum incision that reduces discomfort and facilitates rapid healing. As such, the incision may be formed as Thomas teaches, by delivering the grommet on a sharpened carry-through rod which forms a pilot opening or piercing through which a beveled grommet end is forced. Alternatively the above bevel design may be modified and extended to form a pointed piercing edge. In the latter configuration the grommet would be carried and delivered on a shortened carrier rod and the distal beveled end of the grommet would be extended and modified to form the piercing tip itself sufficient itself to form the pierced opening.

Another object of the present invention is for the means of rotation to provide a low-silhouette and inconspicuous profile at the front or visible side of the pierced opening, for traditional cosmetic reasons. While introducing a healing implant for an approximately eight-week period it is desirable for the device to conform as much as possible with consumers' traditional habit and practice. As such it is desirable that the means provided for and recommended for regular rotation maintenance, described above, be as diminutive as reasonably possible. During normal wearing of ornamental jewelry consumers typically wear such jewelry next to their body, and adjacent to the pierced opening. It is unreasonable to expect consumers to be satisfied with a piercing healing device that caused their jewelry to conspicuously stand off from their body during the extended eight-week healing phase.

Another object of the invention is to maintain the inserted ornamental jewelry close to the ear so such jewelry does not stand off considerably or noticeably as with prior art. With the present invention the proximal collar may have a relatively narrow cross section since the jewelry is retained within the grommet shaft and not retained by a large external rubber plug and cup assembly as with prior art.

Optimally, a preferred and natural method of rotation would be to grasp the device with opposing force from the index finger and thumb at the distal and proximal ends of the device. Optimally such grasping will have the desired effect of further impinging the rear clasp together with the shaft to function as a unit to easily rotate. It would be further desirable if the distal end of the grommet post and the receiving core of the rear clasp were keyed or otherwise receptive to an enhanced tight fit especially when compressed. It would be further desirable to rotation if the length of the grommet shaft was such that the opposing force upon the distal and proximal ends would not bring them into frictional contact against the front and back of the pierced body member (i.e., earlobe). It would be further advantageous though not essential if the proximal flange end of the piercing healing grommet were shaped with somewhat squared or angular edges rather than circular. Such edges would further facilitate a leverage means during rotation.

Another object of the invention is to provide a piercing grommet system that can be deployed by a quick, easy, sterile method with minimum discomfort. Therefore, with the present invention the grommet implant is delivered into the pierced passageway by first being carried on a shuttle-guidance rod which in one hollow-grommet version may be modified to extend therethrough and, with the beveled sharp edge of the distal end of the hollow grommet, form the pilot piercing tip (as Thomas teaches); or in another version the guidance rod may be foreshortened and the beveled distal edge of the above grommet will have been extended and modified to form the solid leading incision point itself.

Another object of the invention is to retain the inserted conventional earring posts or hoops within the grommet shaft portion with a frictional press fit within at any point or points along the bore of the grommet, and not as in prior art by a rubber plug standing off considerably and noticeably outside the pierced channel. Friction needed to retain the jewelry posts may be provided by the close fit of the soft interior walls of the grommet shaft, or by interference bumps, or by a stepped or wedging bore design, or by an integral thin and flexible membrane extending from the walls of the bore and towards the center such membrane may or may not be scored or pre-cut into sections or flaps, or by other textures or interference shapes that may be provided, or by the particularly tacky selection of material, all such friction promoting shapes either molded integrally with the grommet or separately affixed. In case of a flexible grommet material the friction upon the earring shaft may be indirectly enhanced by the normal compression force of the earlobe or cartilage tissue surrounding the grommet. Optionally, the inserted earring posts may be retained within the grommet by a wedging or “pinching” effect provided by tapering the interior walls of the grommet to form a narrowing channel.

Optionally the earring and the grommet may be retained by a frictional interference designed into the rear clasp or clutch attached to the ornamental earring or hoop.

Another object of the present invention is to provide for positive retention of the grommet at the distal end to further mitigate the potential problem of removing the grommet when removing the interchangeable ornamental earrings. The distal end of the grommet may be tapered and barbed or notched, or may be circumscribed with a channel or groove. Immediately after piercing, and potentially automatically, a specialized washer or “C” shaped clip or clasp, also referred to herein as a backing member, may be slid over or around the distal end of the grommet and be retained by the above means for example, and be designed to fulfill multiple critical purposes as are detailed next. As such it is an additional object of the present invention for the rear clasp that retains the grommet implant to also be designed to actively facilitate healing.

A further object of the invention is to provide for the rear clasp or backing member to serve as a comfort buffer at the back of the ear in the case of an ear piercing, since the consumer will wear the grommet implant device night and day for a recommended eight weeks, with ear piercing applications. As such it would be desirable for a rear clasp to be formed of a comfortable material and contour.

Another aspect of the present invention provides for the rear clasp to be formed of a shape, texture and material that would facilitate being compressively engaged by an opposing force of thumb and index finger to facilitate rotation of the grommet shaft as a unit. As will be described in detail, preferably the inner core or bore of the rear clasp would be designed to be keyed or otherwise conform to the distal shape of the grommet such that when compressively engaged and rotated the grommet and rear clasp hold and move together as a unit, as it would be undesirable during rotation for the rear clasp to rotate freely and independently as with conventional rear clasps.

It is an additional object of the present invention for the rear clasp as well as the grommet implant device to be integrally incorporated with anti-microbial agents or compounds which automatically release therefrom throughout the healing phase. Upon piercing, the rear clasp receives and positively though detachably retains the distal end of the grommet for an approximate eight-week period in the case of ear lobe piercing. Serous bio mass including blood from the incision is certain to be carried by and on the distal end of the grommet and into the rear clasp where it will remain throughout healing. Furthermore, at the end of the healing phase, the grommet implant is intended to be removed and discarded by the consumer. It would be desirable to know that any communicable germs within the rear clutch and grommet would have been subjected to ample germicides.

Another object of the invention is to provide a piercing grommet that is disposable and readily replaceable if necessary during the extended healing process. Special anti-microbial germicides may be incorporated into the resins of the grommet material prior to forming or molding the grommet part and released therefrom automatically. Such germicides remain as active agents for limited time periods which depend partly upon the wall thickness of the grommet which is one determiner of the amount of germicides that may be contained. Thus a disposable and, if necessary, a readily replaceable grommet provides the means of keeping the newly pierced opening clean and maintaining fresh active anti-microbial agents at work in the pierced opening.

Another object of the invention is to provide for replacement healing grommets in the event that the original grommet were lost or for other reason. In such case grommets formed with dull tips may be manually inserted. Further, to facilitate easy insertion and removal, the locking design of such grommets would optimally be of a low profile, such as the circumscribed channel described above. A replacement rear clasp would be provided which as well which would be designed specifically for the replacement grommet.

### BRIEF DESCRIPTION OF THE DRAWINGS

These as well as other features of the present invention will become more apparent upon reference to the drawings.

FIG. 1 is a perspective view of the piercing/healing grommet assembly components, and healing grommet in accordance with a preferred embodiment of the present invention within which is nested a conventional earring also shown;

FIG. 2 is a perspective view of a cannula constructed in accordance with a second preferred embodiment of the present invention wherein there is depicted a cannula lumen with a closed sharpened distal end;

FIG. 3 is a perspective view of a healing grommet constructed in accordance with a fourth preferred embodiment of the present invention wherein is depicted a washer retaining system;

FIG. 4 is a perspective view of a healing grommet constructed in accordance with a third preferred embodiment of the present invention wherein is depicted a grommet with a closed, bulbous distal end;

FIG. 5 is a perspective view of a healing grommet constructed in accordance with a second preferred embodiment of the present invention wherein is depicted a grommet with a hollow trumpet shaped distal end;

FIG. 6 is a perspective view of a healing grommet constructed in accordance with a fifth preferred embodiment of the present invention wherein is depicted a textured shaft for resisting inadvertent removal;

FIG. 7 is a cross sectional view of a healing grommet constructed in accordance with a fifth preferred embodiment of the present invention wherein is depicted a means for impinging a standard earring shaft; and

FIG. 8 is a perspective view of a jaw type “clamp” used to compress the distal ends of replacement healing grommets such as those described in FIGS. 4 and 5.

FIG. 9 is an exploded, cross-sectional view of the healing grommet of the present invention as utilized as part of an earring piercing deployment system, wherein the grommet and the deployment system are constructed in accordance with a preferred embodiment of the present invention.

FIG. 10 is a frontal view taken along line 10-10 of FIG. 9.

FIG. 11 is an exploded, cross-sectional view of a healing grommet as incorporated into an earring piercing system, wherein the grommet and piercing system are constructed in accordance with another preferred embodiment of the present invention.

FIG. 12 is a side perspective view of the healing grommet and piercing system depicted in FIG. 11 wherein said grommet is shown axially disposed through an earlobe and operative to receive an earring therewithin.

FIG. 13 is the side perspective view of FIG. 12 wherein said earring, and more particularly the earring post thereof, is shown received within a healing grommet anchored within the piercing formed within the earlobe.

### DETAILED DESCRIPTION OF THE INVENTION

The detailed description as set forth below in connection with the appended drawings is intended as a description of the presently preferred embodiments of the invention, and is not intended to represent the only form in which the present invention may be constructed or utilized. The description sets forth the functions and sequences of steps for constructing and operating the invention in connection with the illustrated embodiments. It is understood, however, that the same or equivalent functions and sequences may be accomplished by different embodiments and that they are also intended to be encompassed within the scope of this invention.

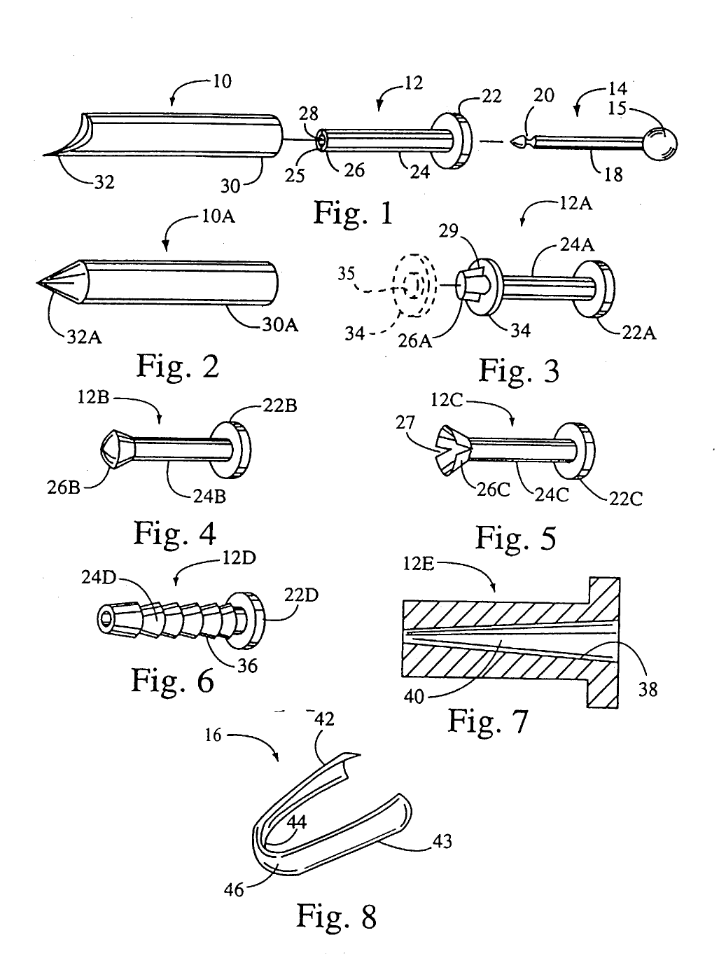

Referring now to the Figures, and initially to FIG. 1, there is shown the components of an improved ear piercing/healing grommet and delivery system. An elongate cylindrical sleeve or healing grommet 12 is formed of a bio-compatible material, such as medical grade plastic. In such embodiment, the sleeve or grommet 12 is specifically designed to embed and reside within the pierced channel opening in the ear. The grommet 12 may be provided with an annular collar or flange 22 formed about the proximal end thereof which serves to radially abut about the tissue surrounding the piercing opening, thereby defining the entryway into the piercing and provide means for anchoring the grommet 12 axially within the formed piercing. As a consequence, grommet 12 will provide the individual with a channel or pathway through which he or she may insert ornamental earrings and the like and change jewelry, which is not available via piercing made directly through flesh insofar as such piercing necessarily require that the tissue heal about such piercing, which can take over a month for initial healing to take place and up to several months for complete healing for most individuals.

In a preferred embodiment the grommet 12 may be formed of a relatively flexible material to thus enable individuals to readily insert and affix earrings or other types of jewelry having arcuate contours, such as hoops and the like. By varying the durometer hardness of various grommet materials, and by varying the thickness of various sections of the grommet 12 advantages may be gained over prior art for achieving vital and specialized tasks. For example, it is well known that intermittent rotation of the designated imbedded piercing shaft (or in this case a healing grommet 12), is critical to successful and timely healing of the pierced opening. Said annular collar or flange 22 may be formed with ample rigidity and thickness for easy grasping or compressively engaging for the above rotation purpose, while still maintaining a relatively thin cross section. Further, the shaft portion 24 of grommet 12 may be of ample length to allow the space needed for easy grasping of said flange 22. As mentioned above, when ornamental earrings such as depicted in FIG. 14 or hoops are inserted into the shaft 24 of grommet 12 said flange 22 will not be noticeable to casual observance as with prior art. Furthermore, said flange 22 may be further camouflaged by forming the flange 22 as clear or translucent or in any number of skin-toned colors.

According to the preferred embodiment, the relatively soft material of said grommet shaft 24 may preferably provide the frictional means to retain the ornamental earring post 18 of earring 14 FIG. 1 with a press fit. Optional means of facilitating a press fit of earring 14 into grommet 12e is described in FIG. 7 wherein the interior walls 38 of the grommet 12e are shown to be tapered to form a wedge effect 40. The normal compressive force of the surrounding earlobe or cartilage flesh about the grommet 12 will further indirectly aid in the desired press fit to retain the shaft 18 of ornamental jewelry 14, and thereby potentially eliminate the need for a standard jewelry “butterfly” clasp. As mentioned, the flexible walls of the healing grommet 12 will also allow for the insertion of curved jewelry such as hoops.

Alternatively, an ornamental earring may be retained within the healing grommet 12 by means of a standard clasp or clutch which may be snapped onto the distal end 20 of earring 14 after said earring 14 is inserted through the imbedded grommet 12. Said use of a standard clasp may serve the dual purpose of simultaneously retaining the grommet 12 in the ear opening.

To facilitate delivery of the novel healing grommet 12 of the present invention there is to be detachably mounted axially about the shaft portion 24 of the grommet 12 a beveled cannula member 10 having a hollow lumen extending there through. When assembled and then deployed the grommet 12 is to be cylindrically encased within the cannula 10. In this regard it is contemplated that when deployed, the cannula 10 will be advanced completely through the earlobe or ear cartilage tissue with the grommet 12 remaining embedded there within by virtue of the anchoring effect provided by the annular collar or flange 22 formed upon the proximal end of such grommet 12. Said pierced opening or pathway is produced by the beveled cutting edge 32 of said cannula 10. Although depicted as a beveled cannula, it should be recognized that cannula member 10 may take any of a variety of conventional cutting needle designs known in the medical arts to produce a clean cutting-edge incision through the ear tissue optimal to rapid healing, such as those produced by Hart Enterprises, Inc. of Wyoming, Mich. or later developed in the art.

Advantageously, unlike prior art systems the cannula is a one-piece seamless cutting device. In contrast to prior art the cannula tip 32 may be ground or formed into any number of angles and shapes optimally customized for cutting pathways through different areas of the ear such as the earlobe or cartilage while avoiding the tearing effect of prior art tips. Advantageously, unlike prior art systems, by using a razor cutting edge the tissue undergoes minimal tearing which is known in the art to occur by blunt or pointed piercing earrings or posts. Such prior art practices are known to produce significant trauma, swelling and recurring inflammation, as well as increase the chance of infection. Additionally, healing times are known to be substantially prolonged due to such prior art techniques.

Trauma is further minimized by virtue of the design of the present invention insofar as the detachable cannula portion 10 is preferably mounted completely about shaft portion 24 of the healing grommet 12 such that the same forms a continuous cylindrical body as the same is advanced through tissue (although it is likewise contemplated that cannula 10 may only cover a portion of shaft 24). Because the cannula member 10 may be hollow throughout there may be disposed within the cannula 10 gauze or the like to insulate and absorb such tissue, blood, etc. The cannula 10 and elongate portion 24 of the healing grommet 12 may additionally be coated or saturated with an anesthetic or anti-microbial agent, or both, or other healing agents as may be necessary for a given application. Although not shown, it is further contemplated that the healing grommet 12 and more particularly the cannula 10 mounted thereon, can minimize trauma even further to the extent the same is deployed such that the cannula 10 is simultaneously rotated while being axially advanced through the target site of tissue. In this regard, it is well known in the art that trauma is minimized to the extent a rotational shearing force is applied to increase a coring effect, if desirable, as has been established in medical biopsy procedures.

In another embodiment of the present invention the cannula 10a depicted in FIG. 2 may be formed similarly to the cannula 10 referenced above, except for having a closed distal end 32a. Advantageously, as with the above cannula member 10, the closed tip version 32 may accommodate any number of designs of sharpened tips customized to optimally cut a pathway through the ear for the same advantages cited above. With the closed tip design 32 the above mentioned tissue and blood cannot enter the cannula 10a from the distal end 32, and the above mentioned gauze or insulating material would not be needed.

Although a delivery device for implanting the cannula/healing grommet piercing assembly 10,12 is not shown it is contemplated that, as with conventional devices, the cannula/healing grommet assembly 10,12 would be forced or shot through the “gun barrel” of a disposable delivery device. Said barrel would be aligned with a cannula “catching” and receiving cartridge integrated into the device and situated on the opposite side of the ear tissue being pierced. Maintaining alignment of the cannula/healing grommet assembly 10,12 within said barrel and also through the targeted ear tissue, may be achieved by modifying the flange 22 of grommet 12 when piercing to take a cylindrical shape to conform to the inside of said delivery barrel. Additionally, the grommet assembly delivery device may provide for any of several means of alignment and guidance of the distal end 32 of the cannula 10. When the delivery barrel of the above device and the receiving cartridge are positively converged thus clamping against the opposite sides of the targeted ear, then alignment may be relatively easily assured. As soon as the cannula tip 32 pierces the ear tissue the cannula tip 32 becomes anchored into an additional point of guidance and alignment.

As will be appreciated, to enhance the safety during utilization of such novel piercing/healing earring system in FIG. 1 above, in particular to isolate the cannula member 10 intended to be mounted thereonto said healing grommet 12, it will be recognized that following the insertion of the healing grommet 12 through a given cross-section 24 of tissue it will be necessary to capture and isolate the cannula member 10 utilized to form the passageway there through. To that end, it is contemplated that a specialized cartridge, such as those disclosed in co-pending patent application Ser. No. 09/264,498, (the teachings of which are expressly incorporated by reference), or other cartridge designed to retain and isolate a spent cannula, facilitate the isolation of the cannula member 10 utilized as part of the piercing process. In this respect, it should be recognized that such cartridge may function merely to isolate the cannula member 10. Said isolation is well known as a requirement under government regulations for handling medical waste commonly dispensed into well known SHARPS containers.

It has been mentioned that during the extended healing phase it is necessary to wear the piercing earring night and day. Said earrings are commonly retained with metal clasps which tend to scratch and irritate end users especially when said metal clasps are pressed against the side of the neck when reclining. Such irritation may be substantially or completely eliminated by the use of the novel healing grommet 12. It is first possible with the present invention to avoid the problem by simply removing the ornamental earring 14 and its accompanying clasp before sleeping. However, when end users prefer to leave ornamental earrings in place when reclining it is important to provide means for avoiding the use of hard-edged rear clasps, traditionally provided by current vendors, to retain the ornamental earring 14 and I or the grommet 12. If the end user consumer desires to leave the earring 14 in place while sleeping, then a soft rear clutch may be employed and attached to the distal end 20 of the ornamental earring 14. Alternatively, to protect the end user during sleep from the pointed end 20 of the earring 14 the retaining washer 34 depicted in FIG. 5 may be formed in a conical shape to insulate the sharp end 20 of the earring 14 while also retaining the grommet 12a at both ends of the pierced channel. Though not shown, another means for retaining the grommet 12 at both ends of the pierced channel, while providing a relatively comfortable backing when the end user is reclining, may be achieved by providing a shaft 24 that is sufficiently long enough to bend or fold after exiting the pierced channel.

In use it will be desirable to prevent the grommet 12 from being inadvertently pulled out of its implant position in the pierced channel when the end user removes the ornamental earring 14. Optional designs will continue to be discussed for positively retaining the grommet 12 at both ends of the pierced channel, and for adding frictional resistance to said removal of the grommet shaft 24 to overcome the degree of press fit used to hold the shaft 18 of earring 14 inside the shaft 24 of grommet 12.

In use, an easily learned practical means of preventing the grommet 12 from being inadvertently removed when removing ornamental jewelry such as 14, will be to simply apply moderate opposing pressure against the flange 22 of grommet 12 when removing said earring 14.

In another grommet embodiment an alternative means of retaining the shaft 18 of ornamental jewelry 14 is depicted in FIG. 1. By designing interior walls 38, FIG. 7 which taper and narrow such as depicted in the cross section of grommet 12e the shaft 18 of earring 14 may be impinged or wedged as it is advanced into the narrowing channel 40 of grommet 12e. Such impingement may provide the further friction press fit needed to retain the earring 14 and to do so advantageously without the need for a standard clasp.

Alternative embodiments of the preferred healing grommet 12 are depicted in FIGS. 3 through 6 depicting novel ways to prevents the grommet 12 from inadvertently slipping out of position once implanted and resting within a given pierced channel, and to also retain said grommet 12 without the need for conventional clasps.

In a preferred alternative grommet embodiment depicted in FIG. 3, a healing grommet is shown to be positively retained with a washer 34. In use, after the shaft of grommet 12a is implanted into the pierced opening washer 34 is to be slid onto and over the barbed distal end 26a of grommet 12a and onto the shaft 24a as depicted in FIG. 3, and said washer 34 will be retained by said barbs 29, and thereby preventing the healing grommet 12a from inadvertent removal. To facilitate sliding the washer 34 over the barbed end 26a and onto the shaft 24a of grommet 12a the inside ring 35 of the washer 34 may be formed of a thinner membrane than the outer section of the washer 34 50 the inner ring 35 may flex and yield as it is pushed over the tapered and barbed end 26a of the grommet shaft 24a. Alternatively, the inner ring 35 may be formed with slots or be otherwise keyed to the distal end 26a so as to freely slide past the end 26a and onto the shaft 24a. In the latter case in use after the washer 34 passed the barbed end the washer 34 would then immediately be rotated a very short distance to pass the keyed “doors” and thereby achieve the reasonably high probability of remaining locked in place on shaft 24a of grommet 12a. In a further alternative version not shown, washer 34 may be formed as a “C” clip designed to potentially automatically slide into a channel slot circumscribing the distal end 26a of the grommet post 24a.

Healing grommets may advantageously be formed to be self anchoring at both ends of the pierced channel. With a one-piece self anchoring version it is contemplated that the distal end of said grommet be integrally formed to have distal ends 26b, 26c of FIGS. 4 and 5 which are of larger diameters in their memory states than their posts 24b, 24c. To provide for easy insertion into the pierced opening the enlarged distal ends 26b, 26c will need to be reduced to a smaller diameter. FIG. 8 depicts one such reducing device which is described below. As described below there is anticipated a need for the end user consumer to replace healing grommets intermittently during the extended healing process, one reason being to introduce fresh anti-microbial agents to the pierced opening. The self anchoring grommet versions depicted in FIGS. 4 and 5 are well suited to insertion by the end user by employing the “jaw” or “V” clamp 16 depicted in FIG. 8, and same are referred to here as replacement grommets. Though not depicted in use replacement grommet/“jaw” clamp assemblies would be provided to the end user.

In said alternative embodiment replacement grommet as depicted in FIG. 4, the bulbous memory state of the distal end 26b is depicted as closed. One potential novel advantage of a closed end 26b design is that said closed end 26b may potentially serve to block or otherwise prevent the hard and pointed distal end 20 of an ornamental earring 14, FIG. 1 from coming into direct contact with the side of the neck and cause the above noted irritation. In a further preferred alternative replacement grommet embodiment depicted in FIG. 5, there is provided a grommet 12c having a memory state taking the shape flared or trumpet-shaped distal end 26c which would impinge against the back side of the ear for the above self-anchoring purposes. The trumpet-shaped distal end 26c of grommet 12c is depicted as hollow throughout both ends of the grommet.

As both alternative replacement grommet embodiments of FIGS. 4 and 5 contain distal ends 26b, 26c which are larger than their respective shafts 24b, 24c, to facilitate insertion of said shafts 24b, 24c, it will be necessary to compress said distal ends 26b, 26c during insertion. In assembly, special fixtures may be readily made to compress the trumpet end 26c of grommet 12c, and the bulbous end 26b of grommet 12b to thus insert same into a “jaw” clamp 16 as depicted in FIG. 8 for storage and later deployment. As mentioned above it may become desirable to replace grommets intermittently during healing for the purpose of introducing fresh anti-microbial agents or for other purposes to enhance healing.

In use the trumpet grommets 12c and the bulbous grommets 12b, may be pre-fitted with an elongated “alligator” jaw or “V” type clamp 16 depicted in FIG. 8. As illustrated, such jaw clamp 16 has first and second arm members 42, 43, that impart an inwardly compressive force. In use, the arms 42, 43 compress about the flared trumpeted portion 26c, of grommet 12c, and compress about the bulbous portion 26b of grommet 12b to thus enable the same to assume a flattened, more radially compressed configuration to thus enable such portions 26c, 26b to be inserted directly through a pre-formed pierced passageway. The trumpet end 26c also depicts relief gaps or slits 27 to serve as gussets and facilitate compressing by the clamp 16 of FIG. 8. In the above scenario, end user customers would simply insert the jaw clamp 16 with the healing grommet engaged therein and pull the clamp 16 off from its exit site, as would occur when the clamp 16 is advanced through a piercing and then when the clamp 16 is removed leaving the grommet 12c in position. Although depicted as a jaw type clamp, it will be readily recognized by those skilled in the art that such clamp 16 may take the form of a conical sleeve or jacket which radially encases the distal end 26b, 26c of the grommets 12b, 12c, such that the same trumpet and bulbous distal ends 26b, 26c are caused to assume such compressed configuration unless otherwise unconstrained.

Grommet 12d of FIG. 6 depicts an alternative embodiment of a novel grommet retaining means for the purpose of further resisting inadvertent removal of the grommet 12 when ornamental earrings such as 14 are removed. The shaft 24d is depicted to be textured with saw tooth barbed ridges 36 oriented to facilitate entry into the pierced opening yet to resist inadvertent exiting from the pierced opening. Such barbs 36 could be alternatively formed as bumps or ridges or a combination of same. It is well known in the industry that the pierced opening conforms to the shape and contour of an implant. For example it is well known that the tissue surrounding pierced openings will close down to the smaller diameter of ornamental earring shafts after having been previously formed with a larger diameter piercing earring shaft. Therefore, the above described textures or barbs 36 will be predictably safe to use as well as effective.

Advantageously, by introducing the grommets of the present invention, there is thus substantially reduced, if not eliminated, the possibility of infection that can occur in those innumerable individuals impatient with the healing process and who want to rapidly change jewelry following the formation of a piercing, and prior to completion of initial healing. As is widely known prematurely changing jewelry following the formation of a piercing greatly increases the chances that an infection can develop at the piercing site, thus significantly prolonging healing time, and increasing customer frustration.

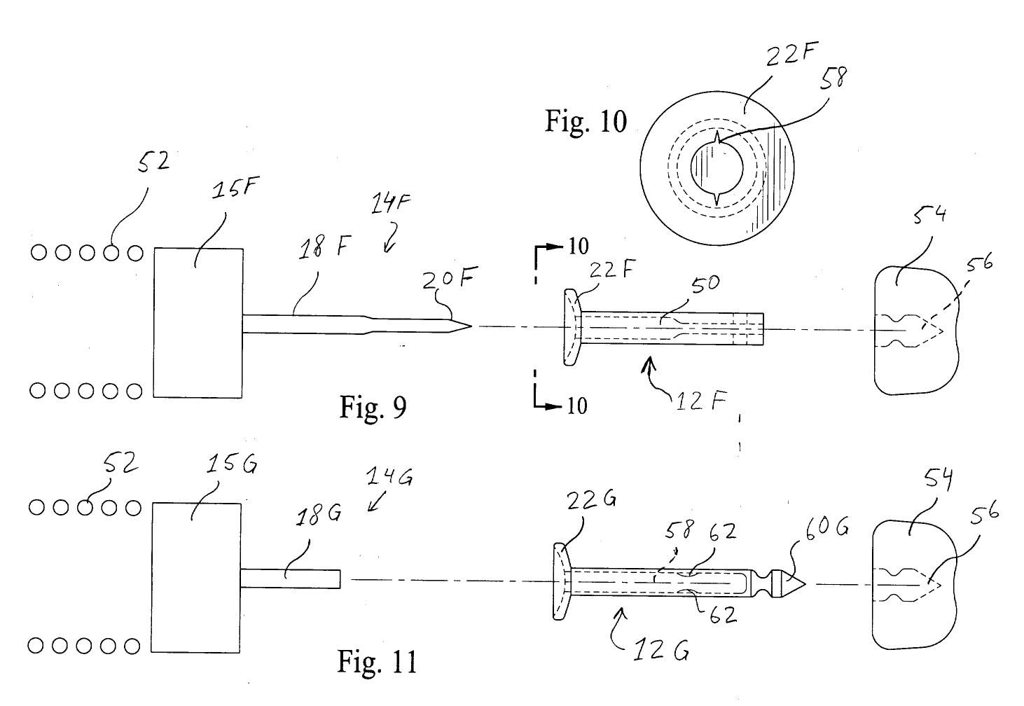

To help further achieve such objectives, there is further disclosed herein systems utilizing the healing grommet of the present invention that are useful in either forming new piercing, or for use in piercings formed by other means, such as conventional piercing practices. Referring now to FIG. 9, there is shown a healing grommet 22F constructed in accordance with yet another preferred embodiment of the present invention for use in forming a piercing with piercing earring 14F, the latter being shown axially aligned with the healing grommet 12F, such piercing earring 14 is advanced therethrough via spring loaded mechanism 52, the latter being exemplary of prior art piercing mechanisms operative to advance the piercing 14F through skin and soft tissue, such as earlobes, belly buttons and the like. In this regard, such piercing earring 14F, and more particularly the piercing element 20F of post 18F thereof, will extend through the lumen 50 of the grommet 12F ultimately into the distal end thereof.

In order to capture and securably hold the piercing earring into position as the same is received through the lumen 50 of grommet 12F, the system further comprises a specialized rear clasp or backing member 54, the latter having a cavity 56 formed thereon for receiving and securably holding the piercing element 20F formed upon the piercing earring 14F. As will be appreciated by those skilled in the art, cavity 56 will be sized and configured to securably interconnect with the post 18F of piercing earring 14F, and more particularly the sharpened distal piercing element 20F thereof, to thus prevent the earring 14F from becoming dislodged within the lumen 50 of the healing grommet 12F. Such backing member 54 further advantageously provides a cushion such that the piercing element 20F formed upon the piercing earring 14F does not inadvertently become compressed against surrounding tissue, such as the neck or base of the skull, which is known in the art to cause substantial pain and irritation following the formation of ear piercings.

To further enhance the ability of the piercing earring 14F to remain secured to the healing grommet 12F, annular collar 22F formed thereon may be provided with generally annular flap portions, such as 58 depicted in FIG. 10, which are operative to impart an axially compressive force about the earring post 18F to thus enable the same to remain firmly lodged within lumen 50 of the healing grommet 12F. In this regard, such flap portions 58, which may be optional, provide yet further means to securably interconnect the healing grommet with the piercing earring such that when coupled with backing member 54, the objectives of the invention can be more readily attained by providing a more comfortable, faster healing piercing that minimizes the risk of infection.

Referring now to FIGS. 11-13, and initially to FIG. 11, there is shown yet another system incorporating the healing grommet of the present invention for use in forming a piercing through skin and soft tissue. As illustrated, the healing grommet 12G is provided with a lumen 58 that is operative to receive an earring post therein. To securably hold such post 18G in position, such lumen 58 may be provided with one or more protuberances 62 that are operative to compressively engage the earring post 18G discussed more fully below. Unlike the other embodiments, however, the healing grommet 12G is provided with a closed distal end having a piercing element 60G formed thereon, the latter being operative to cut through and form a piercing within tissue. Such piercing element will further be configured to be received and interlock within backing member 54, the latter having a cavity 56, as depicted in FIG. 9 operative to receive and engage the piercing element 60G.

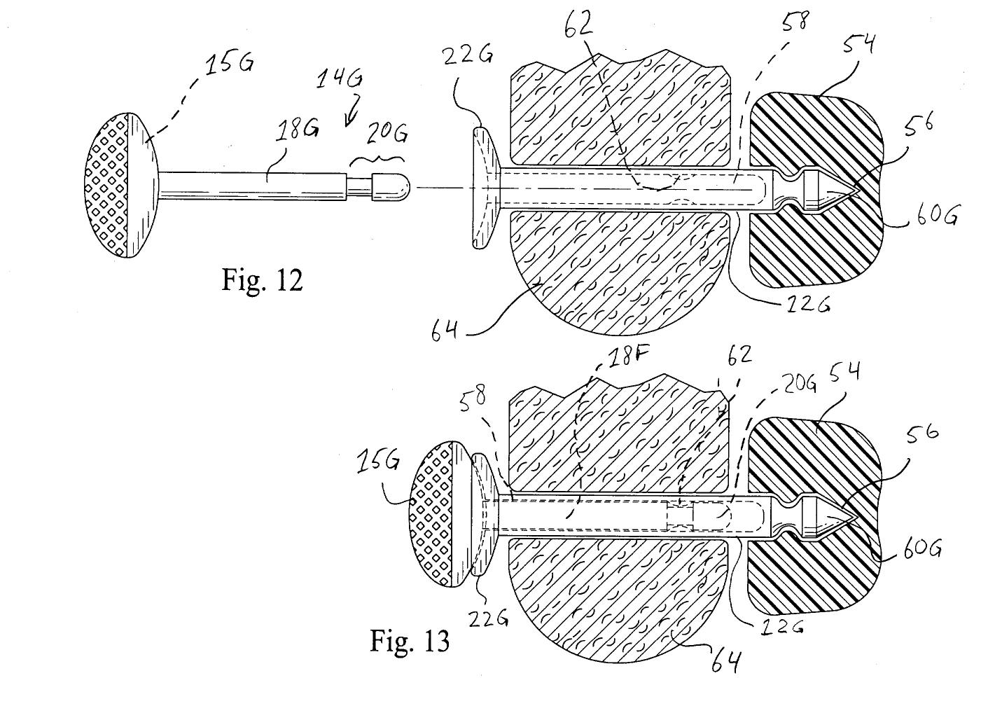

With respect to the formation of a piercing utilizing the embodiment depicted in FIG. 11, it will be recognized by those skilled in the art that the same will be attained by use of conventional piercing mechanisms, such as spring loaded mechanism 52. In this regard, such piercing may first operatively be formed such that the healing grommet 12G is first advanced through the tissue, such as ear lobe 64 depicted in FIG. 12, to thus form the piercing. Along these lines, the piercing will be formed such that the lumen extends through ear lobe 64 such that the piercing element 60G thereof is received within the cavity of backing 54. While in such configuration, the healing grommet 12G will be nested within the piercing and oriented to receive a conventional earring, 14G, such that the post 18G thereof is received within the lumen 58.

To help ensure the secure engagement of the earring 14G within the lumen 58 of healing grommet 12G, protuberances 62 will be operatively positioned to engage with the distal end 20G of post 18G to thus enable the same to be secured therein, as depicted in FIG. 13. As illustrated, distal end 12G is provided with a notched portion, as per conventional earrings, that cooperatively engage with protuberances or formed within lumen 58 to thus enable the same to remain secured therein. Advantageously, the embodiment depicted in FIGS. 11-13 enables a piercing to be formed yet allow conventional earrings to be readily interchanged by the wearer while the healing grommet remains firmly secured into position via the cooperative effect of annular collar 22G and the interconnection between piercing element 60G and backing 54.

As discussed above, by enabling different earrings to be readily worn by individuals having recently had piercings formed within his or her ears it is anticipated to provide substantially higher consumer satisfaction by enabling a variety of earrings to be worn following the formation of a piercing which has not heretofore been available. Additionally, the present invention enables earrings to be readily changed following the recent formation of a piercing that minimizes risk of infection and pain typically associated with conventional practices. Also, as mentioned above, to facilitate healing and/or minimize the pain associated formation of such piercing, it is expressly contemplated that the grommet and backing member components of the present invention may optionally include either an anesthetic and/or antimicrobial agent incorporated therein. Along these lines, such antimicrobial and/or anesthetic agent may take the form of any of a variety of agents that are well-known in the art and may be especially formulated to be incorporated into the materials of the grommet and/or backing member such that the same are slowly released over time to thus impart a therapeutic benefit to the surrounding tissue.

Although the invention has been described herein with specific reference to a presently preferred embodiment thereof, it will be appreciated by those skilled in the art that various modifications, deletions, and alterations may be made to such preferred embodiment without departing from the spirit and scope of the invention.

What is claimed:

1. A jewelry retention system for securing an article within a piercing formed through tissue, the system comprising:
a) a grommet member positionable within said piercing, said grommet having proximal and distal ends and having a lumen extending therethrough defining an axial passageway:
b) a backing member detachably fastenable to said distal end of said grommet member, said backing member being operative to define an anchor for said grommet when said backing member is connected to the distal end thereof.

2. The system of claim 1 wherein said backing member is provided with a cavity operative to receive and interconnect with said distal end of said grommet.

3. The system of claim 2 wherein said cavity formed upon said backing member is further configured to receive a piercing earring post of a piercing earring disposed within and extending from the distal end of said grommet.

4. The system of claim 1 wherein said grommet member further includes an annular collar formed about the proximal end thereof.

5. The system of claim 4 wherein said annular collar of said grommet is provided with at least one retention element for securably retaining an item of jewelry disposed within said lumen of said grommet.

6. The system of claim 1 wherein said distal end of said grommet is closed.

7. The system of claim 6 wherein said distal end of said grommet further includes a piercing element formed thereon, said piercing element being operative to cut and form a piercing through said tissue.

8. The system of claim 7 wherein said backing member includes a cavity operative to securably interconnect with said distal end and said piercing element formed upon said grommet.

9. The system of claim 8 wherein said lumen of said grommet includes at least one protuberance formed therein for securably retaining an item of jewelry disposed therein.

10. The system of claim 9 wherein said protuberance of said lumen of said grommet is operative to securably retain an earring post disposed within said lumen.

11. The system of claim 1 wherein said grommet and said backing member are incorporated with an antimicrobial agent.

12. The system of claim 1 wherein said grommet and said backing member are incorporated with an anesthetic agent.

13. The system of claim 11 wherein said antimicrobial agent comprises a sustained release means.
